(19) United States
(12) Patent Application Publication
(10) Pub. No.: US 2023/0226523 A1
(43) Pub. Date: Jul. 20, 2023

(54) DEHYDROGENATION CATALYST

(71) Applicant: Alan Mitchell Clegg, East London (ZA)

(72) Inventors: Georgievich Dmitri Bessarabov, Potchefstroom (ZA); Phillimon Mokanne Modisha, Potchefstroom (ZA); Cecil Naphtaly Moro Ouma, Potchefstroom (ZA); Kingsley Onyebuchi Obodo, Potchefstroom (ZA)

(21) Appl. No.: 18/119,589

(22) Filed: Mar. 9, 2023

Related U.S. Application Data
(63) Continuation of application No. PCT/IB2021/058308, filed on Sep. 13, 2021.

(30) Foreign Application Priority Data
Sep. 11, 2020 (ZA) 2020/01578

(51) Int. Cl. B01J 23/42; B01J 21/06; B01J 21/08; B01J 35/04; B01J 35/08

(52) U.S. Cl. CPC B01J 23/42; B01J 21/08; B01J 21/063; B01J 35/04; B01J 35/08

(57) ABSTRACT

This invention pertains to a dehydrogenation catalyst. More particularly, but not exclusively, this invention pertains to dehydrogenation catalysts comprising platinum, platinum silicide and/or platinum phosphide being supported on various metal-oxide supports, which may also be modified metal-oxide supports, for the dehydrogenation of a liquid organic hydrogen carrier.

### PRIORITY

This application is a continuation of, and claims priority to, PCT/IB2021/058308 with an international filing date of Sep. 13, 2021, entitled DEHYDROGENATION CATALYST, which claims priority to South African Patent Application 2020/01578 filed on Sep. 11, 2020, the contents of which are hereby incorporated by reference in their entireties.

### FIELD OF THE INVENTION

This invention pertains to a dehydrogenation catalyst. More particularly, but not exclusively, this invention pertains to a dehydrogenation catalyst for the dehydrogenation of a liquid organic hydrogen carrier. The invention also relates to a method of preparing the dehydrogenation catalyst.

### BACKGROUND

Liquid Organic Hydrogen Carrier (LOHC) technology is an attractive technology for long-distance transport and long-term storage of hydrogen. LOHC technology comprise a two-step cycle. The first step comprises loading hydrogen into a LOHC molecule; i.e. a hydrogenation step. Hydrogen is covalently bound to the LOHC molecule during the hydrogenation step. The second step comprises unloading of hydrogen from the LOHC molecule to which it was bound during the preceding hydrogenation step; i.e. a dehydrogenation step.

The LOHC molecule is typically an unsaturated organic compound. Several organic compounds have been explored as suitable LOHC molecules. These include, but are not limited to N-ethylcarbazole, toluene, dibenzyltoluene, benzene, and naphthalene.

Platinum, palladium, ruthenium, nickel and copper include some of the well-known catalysts for the dehydrogenation reaction. A noble metal is typically deposited in small quantities (e.g. 0.3 - 0.5 wt%) on a porous metal-oxide supports such as SiO2, A12O3, TiO2 and V2O5 to produce a noble-metal-containing dehydrogenation catalyst.

Despite continued advancements in the selection and preparation of dehydrogenation catalysts, the known dehydrogenation catalysts suffer from efficiency and stability issues. These issues are typically exacerbated during the prolonged dehydrogenation reactions of LOHC technologies.

### OBJECT OF THE INVENTION

It is accordingly an object of the present invention to provide a dehydrogenation catalyst which overcomes, at least partially, the abovementioned problems and/or which will be a useful alternative to existing dehydrogenation catalysts.

### SUMMARY

According to a first aspect of the present invention, there is provided a dehydrogenation catalyst comprising platinum silicide supported on a metal-oxide support.

The metal-oxide support may take the form of a pellet.

The metal-oxide support may be selected from any one of the group consisting of SiO2, A12O3, TiO2 and V2O5.

The platinum loading of the dehydrogenation catalyst may be between 0.5 and 2.5 wt%.

The silicon loading of the dehydrogenation catalyst may be between 0.1 and 1 wt%.

The molar ratio of silicon to platinum in the dehydrogenation catalyst may be between 1:10 and 1:3.

According to a second aspect of the present invention, there is provided a dehydrogenation catalyst comprising platinum phosphide supported on a metal-oxide support.

The metal-oxide support may take the form of a pellet.

The metal-oxide support may be selected from any one of the group consisting of SiO2, A12O3, TiO2 and V2O5.

The platinum loading of the dehydrogenation catalyst may be between 0.5 and 2.5 wt%.

The phosphorus loading of the dehydrogenation catalyst may be between 0.1 and 1 wt%.

The molar ratio of phosphorus to platinum in the dehydrogenation catalyst may be between 1:10 and 1:3.

According to a third aspect of the present invention, there is provided a dehydrogenation catalyst comprising platinum supported on a modified metal-oxide support, the metal-oxide support having been modified with silicon.

The metal-oxide support may take the form of a pellet.

The metal-oxide support may be selected from any one of the group consisting of SiO2, A12O3, TiO2 and V2O5.

The platinum loading of the dehydrogenation catalyst may be between 0.5 and 2.5 wt%.

The silicon loading of the dehydrogenation catalyst may be between 0.1 and 1 wt%.

The molar ratio of silicon to platinum in the dehydrogenation catalyst may be between 1:10 and 1:3.

According to a fourth aspect of the present invention, there is provided a dehydrogenation catalyst comprising platinum supported on a modified metal-oxide support, the metal-oxide support having been modified with phosphorus.

The metal-oxide support may take the form of a pellet.

The metal-oxide support may be selected from any one of the group consisting of SiO2, A12O3, TiO2 and V2O5.

The platinum loading of the dehydrogenation catalyst may be between 0.5 and 2.5 wt%.

The phosphorus loading of the dehydrogenation catalyst may be between 0.1 and 1 wt%.

The molar ratio of phosphorus to platinum in the dehydrogenation catalyst may be between 1:10 and 1:3.

The metal-oxide supports of all of the above-discussed catalysts may take various shapes and sizes. For example, the metal-oxide supports may take the shape of rods, spheres, plates, foams and honeycombs.

According to a fifth aspect of the present invention, there is provided for the use of any one of the catalysts described herein in a dehydrogenation reaction.

There is provided for the dehydrogenation reaction to be a dehydrogenation reaction of a liquid organic hydrogen carrier to form hydrogen gas.

### BRIEF DESCRIPTION OF THE DRAWINGS

The invention will now be described further, by way of example only, with reference to the accompanying drawings wherein:

FIG. 1 is a graph showing calculated methylcyclohexane dehydrogenation energy as a function of silicon loading (wt%);

FIG. 2 is a graph showing calculated methylcyclohexane dehydrogenation energy as a function of phosphorous loading (wt%); and

FIG. 3 is a graph showing calculated methylcyclohexane dehydrogenation energy as a function of sulphur loading (wt%).

### DETAILED DESCRIPTION

The dehydrogenation catalyst comprising platinum silicide supported on a metal-oxide support is prepared by adding sodium borohydride to a solution containing H2PtC16 and methoxytrimethylsilane to produce platinum silicide (Pt-Si) compounds. The platinum silicide compounds are subsequently mixed with water to form an aqueous suspension of platinum silicide compounds. This aqueous suspension of platinum silicide compounds is then sprayed onto an external surface of various metal-oxide supports. The various metal-oxide supports includes SiO2, A12O3, TiO2 and V2O5.

It will be appreciated that the aqueous solution of platinum silicide compounds may be applied to a metal-oxide support by other means, including chemical vapour deposition, ionexchange, and impregnation. It is further envisaged that the aqueous suspension of platinum silicide compounds may be applied to the external surface of the metal-oxide support by means of chemical vapour deposition.

Instead of a metal-oxide support, the platinum silicide compounds may also be applied to a graphene support.

During the preparation of the above catalyst, the silicon loading range on the metal-oxide support is between 0.1 and 1 wt%. The platinum loading range on the metal-oxide support is between 0.5 and 2.5 wt%. Furthermore, the stoichiometric ratio of silicon/platinum are carefully controlled to avoid deactivation or poisoning of the catalyst. Here, the minimum ratio of silicon/platinum is 1:10 and the maximum ratio of silicon/platinum is 1:3.

The dehydrogenation catalyst comprising platinum phosphide (Pt-P) supported on a metal-oxide support is prepared by mixing phosphoric acid and H2PtCl6 to form a working solution. It will be appreciated that phosphonic acid or sodium phosphate can also be used instead of phosphoric acid. The working solution is then subjected to microwave radiation to produce a platinum phosphide compound. Instead of microwave radiation, the working solution may also be subjected to a pyrolysis process to produce platinum phosphide compounds. The platinum phosphide compounds are subsequently mixed with water to form an aqueous solution of platinum phosphide compounds. The aqueous solution of platinum phosphide compounds is then sprayed onto an external surface of various metal-oxide supports. The metal-oxide supports to which the aqueous solution of platinum phosphide compounds is applied includes SiO2, A12O3, TiO2 and V2O5.

It will be appreciated that the aqueous solution of platinum phosphide compounds may be applied to a metal-oxide support by other means, including chemical vapour deposition, ionexchange, impregnation is further envisaged that the aqueous suspension of platinum silicide compounds may be applied to the external surface of the metal-oxide support by means of chemical vapour deposition.

Instead of a metal-oxide support, the platinum phosphide compounds may also be applied to a graphene support.

During the preparation of the above catalyst, the phosphorous loading range on the metal-oxide support is between 0.1 and 1 wt%. The platinum loading range on the metal-oxide support is between 0.5 and 2.5 wt%. Furthermore, the stoichiometric ratio of phosphorous/platinum are carefully controlled to avoid deactivation or poisoning of the catalyst. Here, the minimum ratio of phosphorous/platinum is 1:10 and the maximum ratio of phosphorous/platinum is 1:3.

The dehydrogenation catalyst comprising platinum supported on a metal-oxide support which has been modified with silicon is prepared by, firstly, hydrolysing alkoxy groups of an alkoxysilane to form silanol. Silanol is then applied to the surface of the various metal-oxide supports. This is followed by a condensation step to form oligomers. During the condensation step, the oligomers form a hydrogen bond with hydroxyl groups of the metal-oxide support. Here, a covalent linkage is formed with the metal-oxide support by concomitant loss of water due to drying. The silanes can also form self mono-assembly at the metal-oxide support by solution or vapor phase deposition processes.

An illustration of the above-described salination process of the metal-oxide support is shown below:

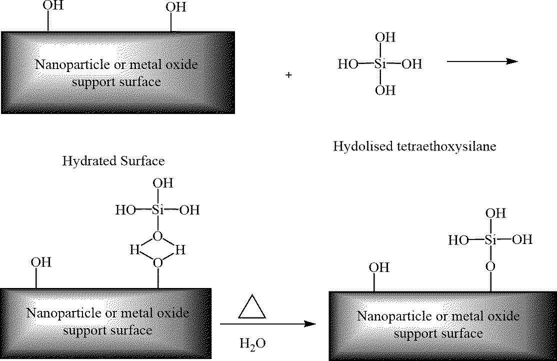

The modified metal-oxide support is then impregnated with a solution of H2PtCl6 and subjected to a calcination step in air at a temperature of from 350 to 650 degree Celsius for a period of 1 to 10 hours and a reduction step with hydrogen gas.

During the preparation of the above catalyst, the silicon loading range on the metal-oxide support is between 0.1 and 1 wt%. The platinum loading range on the modified metal-oxide support is between 0.5 and 2.5 wt%. Furthermore, the stoichiometric ratio of silicon/platinum are carefully controlled to avoid deactivation or poisoning of the catalyst. Here, the minimum ratio of silicon/platinum is 1:10 and the maximum ratio of silicon/platinum is 1:3.

The dehydrogenation catalyst comprising platinum supported on a metal-oxide support which has been modified with phosphorous is prepared by reacting hydroxyl groups on the surface of the metal-oxide support with phosphorous-containing groups. For example, with the —POOH acid group of phosphonic acid or with the —PO(OH) group of phosphoric acid. On the Lewis acidic metal oxide surfaces, binding originates from initial coordination of the phosphoryl oxygen atom (P═O) to a Lewis acidic site on the surface of the metal-oxide support. As a consequence of the afore, the phosphorous atom becomes more electrophilic and induces the consecutive heterocondensation with the neighbouring surface hydroxy groups, resulting in a strong covalent bonding of P—O—M. An illustration of the afore is shown in the below diagram:

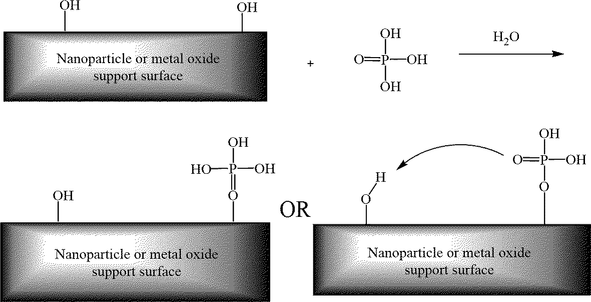

The phosphorous modified metal-oxide described above is then impregnated with a solution of H2PtCl6 and subjected to a calcination step in air at a temperature of 350 to 650° C. for a period of 1 to 10 hours and a reduction step with hydrogen gas.

During the preparation of the above catalyst, the phosphorous loading range on the metal-oxide support is between 0.1 and 1 wt%. The platinum loading range on the modified metal-oxide support is between 0.5 and 2.5 wt%. Furthermore, the stoichiometric ratio of phosphorous/platinum are carefully controlled to avoid deactivation or poisoning of the catalyst. Here, the minimum ratio of phosphorous/platinum is 1:10 and the maximum ratio of phosphorous/platinum is 1:3.

### SPECIFIC EXAMPLES

Dehydrogenation of methylcyclohexane on Si, P and S modified Pt surfaces:

Methylcyclohexane was used for illustration purpose only. Other chemically similar aliphatic hydrocarbons such as perhydrodibenzyltoluene, perhydrobenzyltoluene, etc could also have been used.

The effect of additives on Pt surfaces was investigated using ab initio density functional theory (“DFT”).

The reaction energy for the dehydrogenation of methylcyclohexane was calculated using ab initio DFT at different weight percentages of the additives (Si, P and S).

Pt surface slabs were created from bulk Pt and the additives added to the active sites of the surface at different concentrations. The choice of additive addition was informed by Monte Carlo configuration search that took into account all possible sites the additive could attach.

The calculated dehydrogenation energy on pristine Pt (111) surface was 73.09 kJ/mol. This is in close agreement with other reported studies that have reported this energy to be 68.3 kJ/mol. Upon additive addition, a reduction in the dehydrogenation energy was observed. The reduction in the dehydrogenation energy was as much as 64 percentage, depending on the weight percentage of the additive.

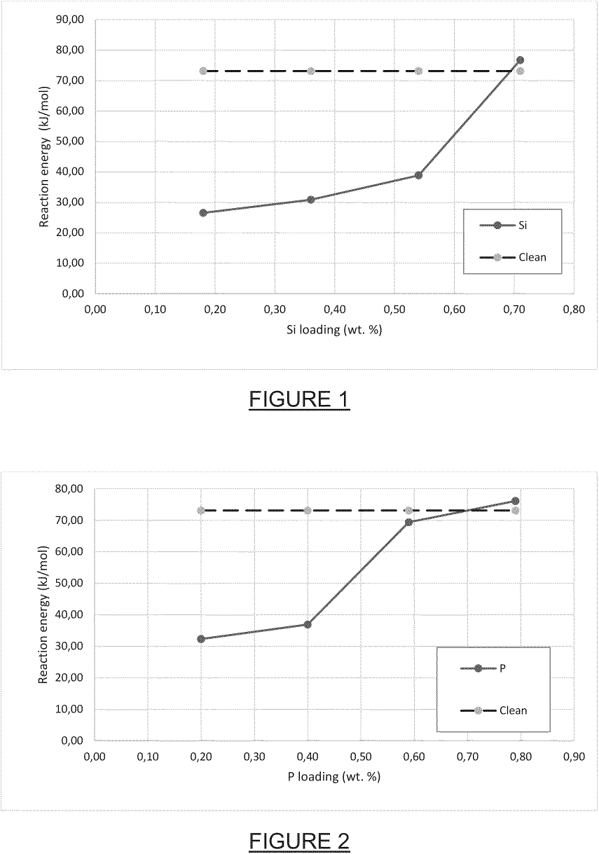

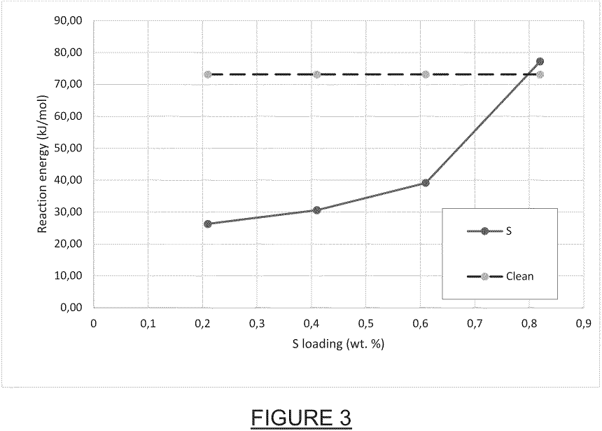

A common observation among all the plots for reaction energy vs additive weight percentage (FIGS. 1 to 3) is that as the concentration of the additive increases, the calculated reaction energies approaches that of pristine Pt (111) surface.

From the Figures, it is evident that additive concentration in the range of ~0.2 - 0.7 weight percentage on the Pt (111) surface lowers the dehydrogenation reaction energies. When the additive concentration is above 0.7 weight percentage the calculated dehydrogenation energy is almost equal to that of pristine Pt surface.

It will be appreciated by those skilled in the art that the invention is not limited to the precise details as described herein and that many variations are possible without departing from the scope and spirit of the invention.

The description is presented in the cause of providing what is believed to be the most useful and readily understandable description of the principles and conceptual aspects of the invention. In this regard, no attempt is made to show and/or describe structural details of the invention in more detail than is necessary for a fundamental understanding of the invention. The words used should therefore be interpreted as words of description rather than words of limitation.

What is claimed is:

1. A dehydrogenation catalyst comprising platinum silicide or platinum supported on a metal-oxide support.

2. The dehydrogenation catalyst according to claim 1, wherein the metal-oxide support is selected from any one of the group consisting of SiO₂, Al₂O₃, TiO₂ and V₂O_{5.}

3. The dehydrogenation catalyst according to claim 1, wherein the platinum loading of the dehydrogenation catalyst is between 0.5 and 2.5 wt%.

4. The dehydrogenation catalyst according to claim 1, wherein the silicon loading of the dehydrogenation catalyst is between 0.1 and 1 wt%.

5. The dehydrogenation catalyst according to claim 1, wherein the molar ratio of silicon to platinum in the dehydrogenation catalyst is between 1:10 and 1:3.

6. The dehydrogenation catalyst, according to claim 1, wherein the metal-oxide support is in the shape of any one of more of pellets, rods, spheres, plates, foams and honeycombs.

7. Use of the catalyst according to claim 1 in a dehydrogenation reaction.

8. The dehydrogenation catalyst according to claim 1, wherein the phosphorus loading of the dehydrogenation catalyst is between 0.1 and 1 wt%.

9. The dehydrogenation catalyst according to claim 1, wherein the molar ratio of phosphorus to platinum in the dehydrogenation catalyst is between 1:10 and 1:3.

10. A dehydrogenation catalyst comprising platinum supported on a modified metal-oxide support, the metal-oxide support having been modified with silicon or phosphorus.

11. The dehydrogenation catalyst according to claim 10, wherein the metal-oxide support is selected from any one of the group consisting of SiO₂, Al₂O₃, TiO₃ and V₂O_{5.}

12. The dehydrogenation catalyst according to claim 10, wherein the platinum loading of the dehydrogenation catalyst is between 0.5 and 2.5 wt%.

13. The dehydrogenation catalyst according to claim 10, wherein the silicon loading of the dehydrogenation catalyst is between 0.1 and 1 wt%.

14. The dehydrogenation catalyst according to claim 10, wherein the molar ratio of silicon to platinum in the dehydrogenation catalyst is between 1:10 and 1:3.

15. The dehydrogenation catalyst according to claim 10, wherein the phosphorus loading of the dehydrogenation catalyst is between 0.1 and 1 wt%.

16. The dehydrogenation catalyst according to claim 10, wherein the molar ratio of phosphorus to platinum in the dehydrogenation catalyst is between 1:10 and 1:3.

17. The dehydrogenation catalyst, according to claim 10, wherein the metal-oxide support is in the shape of any one of more of pellets, rods, spheres, plates, foams and honeycombs.

18. Use of the catalyst according to claim 10 in a dehydrogenation reaction.

19. The use according to claim 18, wherein the hydrogenation reaction is a dehydrogenation reaction of a liquid organic hydrogen carrier to form hydrogen gas.
